(19) United States
(12) Patent Application Publication
(10) Pub. No.: US 2012/0041058 A1
(43) Pub. Date: Feb. 16, 2012

(54) COMPOSITION OF TRADITIONAL CHINESE MEDICINE FOR REDUCING BLOOD FAT AND PREPARATION METHOD THEREOF

(75) Inventors: Kexuan Tang, Shanghai (CN); Yuliang Wang, Shanghai (CN); Hailiang Xin, Shanghai (CN); Luoou Tang, Shanghai (CN)

(73) Assignee: ANJI GREEN LIFE BIOTECH CO., LTD., Huzhou (CN)

(21) Appl. No.: 13/203,949

(22) PCT Filed: Mar. 9, 2010
(86) PCT No.: PCT/CN2010/070921

§ 371 Date: Aug. 30, 2011

(30) Foreign Application Priority Data
Mar. 26, 2009 (CN) 200910048229.1

(51) Int. Cl. A61K 31/357; A61P 3/06

(52) U.S. Cl. 514/452

(57) ABSTRACT

Composition of traditional Chinese medicine for reducing blood lipid with components of the crude drug of artemisinins and the crude drug of ursolic acid, wherein the ratio of the crude drug of artesunate and the crude drug of ursolic acid is 1:25-25:1 by weight. The preparation method of such composition includes the following steps: the crude drug of artesunate and the crude drug of ursolic acid are mixed at the weight ratio mentioned above, comminuted, then screened through 120 mesh sieve to obtain the composition; or the crude drug of artesunate and the crude drug of ursolic acid are weighed at the weight ratio mentioned above, comminuted, screened through 120 mesh sieve separately, then mixed and screened for three times to obtain the composition. The composition of the present invention can be formulated into formulations for injection, oral administration with pharmaceutically acceptable carriers, and can be used for preparing medicaments for reducing blood lipid.

### FIELD OF INVENTION

The present invention relates to a traditional Chinese medicine composition in the pharmaceutical technology field and the preparation method thereof, particularly to a traditional Chinese medicine composition for reducing blood lipid and the preparation method thereof.

### BACKGROUND OF INVENTION

At present, the number of the elderly having hyperlipaemia is increasing. Since hyperlipaemia and the resultant atherosclerosis readily cause some disease conditions of serious health hazards such as coronary artery disease, stroke and the like, the improvement of the hyperlipaemia condition has become a focus drawing great attention of the medicine researchers. Thus, the development of drugs for reducing blood lipid has become a hotspot in the pharmaceutical industries. Currently, the blood-lipid-lowering drugs for clinical application mainly include: (1) Statins: lovastatin, simvastatin, etc.; (2) Acyl-coenzyme A cholesterol acyltransferase inhibitors: Avasimibe; (3) Fibrates: fenofibrate, gemfibrozil; (4) Nicotinic acid and its derivatives: niacin, acipimox; (5) cholesterol absorption prevention drugs: mostly anion exchange resin. However, most of the above-mentioned drugs still have some shortcomings such as high prices, obvious side effects and rebound after the administration stops. Therefore, the development of the inexpensive, safe and effective blood-lipid-lowering drugs has a broad prospect.

Artemisinin is a sesquiterpene lactone having a new structure firstly extracted from Artimisia annua Linn by Chinese scientists in 1971. Based on its structure, a series of its derivatives such as dihydroartemisinin, artesunate, artemether, etc. had been developed. Compounds of this class have an excellent antimalarial effect. Artesunate is one of the commonly used artemisinin derivatives, which has a better antimalarial efficacy and a better water-solubility and is easier to constitute medicine as compared with artemisinin. Currently, there are some studies showing that artemisinin and its derivatives have unique anti-tumor mechanism, which can selectively kill tumor cells with only a slight effect on normal cells, and have no cross-resistance with the traditional chemotherapy drugs. Artemisinin and its derivatives can also reverse the tumor cell multidrug resistance by inhibiting glutathione transferase activity, and their applications together with traditional chemotherapy drugs can bring a synergistic effect. Ursolic acid is a triterpenoid compound appearing in some natural plants. Some studies have shown that ursolic acid has multiple biological activities such as calming, anti-inflammatory, antibacterial, anti-diabetic, anti-ulcer, reducing blood sugar, etc. In recent years, it is also found that ursolic acid further has the effects of anti-carcinogenic, anti-tumor promoters, and inducing F9 teratoma cell differentiation and anti-angiogenic.

It has been found in the literature searches of the prior art that, in WANG Weidong's paper entitled “The cost-effectiveness analysis for five drugs in treating hyperlipidemia”, Proceeding of Clinical Medicine, 2006, Vol. 15, No. 3, p. 203-205, five drugs including lovastatin, simvastatin, atorvastatin, fenofibrate and Xuezhikang were compared, and the result indicated that simvastatin had a best cost-effectiveness, but the cost is still as high as 173.88 RMB/8 weeks. Jin Hui, et al, in the paper entitled “The adverse effects of simvastatin and its control”, Adverse Drug Reactions Journal, 2006, Vol. 8, No. 2, p. 113-115, reviewed the multiple adverse reactions of simvastatin such as resulting in rhabdomyolysis, liver damage and nervous system damage, etc. From the above literatures, it is known that, although the production of simvastatin has been manufactured locally, its use cost is still a great financial burden for patients who need a long-term use. Despite of a low incidence of its various side-effects, the long-term large-scale use of simvastatin still has some safety risks.

### SUMMARY OF THE INVENTION

The object of the present invention is to provide a blood-lipid-lowering medicine composition and the preparation method thereof, which is able to overcome the shortcomings of the existing technologies. The composition has a high efficiency at a lower dose with a low toxicity, which can significantly reduce the blood lipid. The present invention can be formulated together with pharmaceutically acceptable excipient carriers to form modern formulations such as oral or injection administration dosage forms.

The present invention is achieved through the following technical solutions:

The traditional Chinese medicine composition for reducing blood lipid according to the present invention comprises artemisinin derivatives API (Active Pharmaceutical Ingredient) and ursolic acid API, wherein the weight ratio of artesunate API to ursolic acid API is from 1:25 to 25:1.

Said artemisinin derivatives API refers to one selected from the group consisting of artemisinin, dihydroartemisinin, artesunate, artemether, and the combination of two or more thereof;

Said ursolic acid API refers to ursolic acid.

The weight ratio of artesunate API to ursolic acid API is preferably 1:1.

The method for preparing the above blood-lipid-lowering traditional Chinese medicine composition according to the present invention includes the following steps: mixing artemisinin derivatives API and ursolic acid API at a weight percentage ratio; comminuting the mixture, screening through a 120 mesh sieve; then adding suitable accessories to get a clinically acceptable formulation. Alternatively, the method includes the following steps: weighing artemisinin derivatives API and ursolic acid API at a weight percentage ratio; comminuting them respectively, screening through a 120 mesh sieve; then mixing and screening for three times to obtain the composition.

The present invention has the following beneficial effects: the composition of the present invention has a significant blood-lipid-lowering effect. Moreover, both artesunate API and ursolic acid API have wide sources and low prices. From the market prices of the bulk drugs of the two components, the estimated cost of a day medication is less than RMB 1 yuan. In addition, the composition has a higher security, and thus has a broad market prospect.

### DETAILED DESCRIPTION OF EMBODIMENT

The following embodiments are set forth for illustrating the invention in detail. The detailed examples and specific processes are carried out based on the solutions of the present invention, but the scope for protection of this invention are not limited to the following examples.

### Example 1

### The Treatment Efficiency of Blood-Lipid-Lowering Traditional Chinese Medicine Compositions Having Different Component Proportions on the Acute High-Lipid Mouse Models Resulted from Egg Yolk Emulsion Injection

Male ICR mice of 20 g˜22 g are divided into several groups after 5 d adaptive feeding: control group, fed with normal saline; model group, fed with normal saline; positive control group, fed with Xuezhikang of 1000 mg/kg.

Artesunate group: artesunate, 2 mg/mouse;

Ursolic acid group: ursolic acid, 2 mg/mouse;

Artemisinin group: artemisinin, 2 mg/mouse;

Compound group A: artesunate+ursolic acid, (50 mg+2 mg)/mouse;

Compound group B: artesunate+ursolic acid, (20 mg+2 mg)/mouse;

Compound group C: artesunate+ursolic acid, (2 mg+2 mg)/mouse;

Compound group D: artesunate+ursolic acid, (2 mg+20 mg)/mouse;

Compound group E: artesunate+ursolic acid, (2 mg+50 mg)/mouse;

Compound group F: artemisinin+ursolic acid, (2 mg+2 mg)/mouse;

Dose volume: 0.2 ml, 1 time/d, continuous administration for 14 d; fasting but without forbidding water from the 14^{th} d of administration; at 2^{nd} hour from the last administration, the control group was intraperitoneal injectioned (ip) with normal saline of 0.5 mL/mouse, with the other groups were intraperitoneal injectioned with 75% egg yolk emulsion of a dose of 0.5 mL/mouse, wherein 75% refers to the volume ratio (v/v) of an egg yolk emulsion, thus resulting in an experimental hyperlipidemia. At 20^{th} hour after the injection, collecting blood from the orbital venous plexus of the mouse, so as to determine the serum cholesterol (T-cho) and triglycerides (Trig), the results are shown in Table 2. The reduction rates of cholesterol and triglycerides were calculated, and the results were shown in Table 2, wherein all of the experimental data obtained are expressed in form of χ±SD, and the differences between the groups were tested via the variance analysis by using the software SPSS of version 10. The results indicated that the after two weeks of oral administration, neither ursolic acid nor artesunate had the blood-lipid-lowering effect on the mouse model of high blood lipid caused by the egg yolk emulsion, whereas all of the compounds of artesunate+ursolic with different proportions had significant blood-lipid-lowering effects, such an effect was not achievable for independent ursolic acid or artesunate.

TABLE 1

| | |
|---|---|
| Cholesterol reduction | (Cholesterol of model mouse group − Cholesterol |
| rate (%) = | of administration mouse group)/ |
| | Cholesterol of model mouse group × 100% |
| Triglyceride reduction | (Triglyceride of model mouse group − |
| rate (%) = | Triglyceride of administration mouse group)/ |
| | Triglyceride of model mouse group × 100% |

TABLE 2
Influences of artesunate, ursolic acid and their compounds on the
cholesterol and triglyceride of acute high-lipid mouse model

| | | Number of | | |
|---|---|---|---|---|
| | | animals | T-cho | Trig |
| Group | Dose | (n) | (mmol/L) | (mmol/L) |
| Blank group | — | 10 | 4.11 ± 0.56 | 3.05 ± 0.75 |
| Model group | — | 10 | 8.14 ± 2.42 | 10.22 ± 6.08 |
| Xuezhikang | 1000 mg/kg | 10 | 5.85 ± 1.64* | 5.82 ± 2.61* |
| Ursolic acid | 2 mg/mouse | 10 | 9.11 ± 1.49 | 13.37 ± 3.63 |
| Group | | | | |
| Artesunate | 2 mg/mouse | 10 | 8.32 ± 1.09 | 10.72 ± 2.68 |
| group | | | | |
| Artemisinin | 2 mg/mouse | 10 | 6.10 ± 3.20* | 8.90 ± 4.00 |
| group | | | | |
| Compound | (50 mg + | 10 | 5.17 ± 1.65* | 6.88 ± 2.83 |
| group A | 2 mg)/mouse | | | |
| Compound | (20 mg + | 10 | 5.44 ± 2.50* | 5.75 ± 1.09* |
| group B | 2 mg)/mouse | | | |
| Compound | (2 mg + | 10 | 4.33 ± 1.84* | 5.32 ± 2.12* |
| group C | 2 mg)/mouse | | | |
| Compound | (2 mg + | 10 | 6.01 ± 1.32 | 5.82 ± 2.54* |
| group D | 20 mg)/mouse | | | |
| Compound | (2 mg + | 10 | 5.15 ± 1.12* | 6.54 ± 2.23 |
| group E | 50 mg)/mouse | | | |
| Compound | (2 mg + | 10 | 5.47 ± 3.18* | 7.00 ± 6.02 |
| group F | 50 mg)/mouse | | | |

Note:
*p < 0.05;
**p < 0.01, as compared with the model group.

TABLE 3
Influences of artesunate, ursolic acid and their compounds on the
cholesterol and triglyceride reduction rates (%) of acute high-lipid
mouse model

| | | Number | | |
|---|---|---|---|---|
| | | of | Reduction | Reduction |
| | | animals | rate of | rate of |
| Group | Dose | (n) | cholesterol | triglyceride |
| Blank group | — | 10 | — | — |
| Model group | — | 10 | 0 | 0 |
| Xuezhikang | 1000 mg/kg | 10 | 28.13 | 43.05 |
| Ursolic acid Group | 2 mg/mouse | 10 | −11.92 | −30.82 |
| Artesunate group | 2 mg/mouse | 10 | −2.21 | −4.89 |
| Artemisinin group | 2 mg/mouse | 10 | 25.1 | 12.9 |
| Compound group A | (50 mg + | 10 | 36.49 | 32.68 |
| | 2 mg)/mouse | | | |
| Compound group B | (20 mg + | 10 | 33.17 | 43.74 |
| | 2 mg)/mouse | | | |
| Compound group C | (2 mg + | 10 | 46.81 | 47.95 |
| | 2 mg)/mouse | | | |
| Compound group D | (2 mg + | 10 | 26.17 | 43.05 |
| | 20 mg)/mouse | | | |
| Compound group E | (2 mg + | 10 | 36.73 | 36.01 |
| | 50 mg)/mouse | | | |
| Compound group F | (2 mg + | 10 | 32.8 | 31.5 |
| | 50 mg)/mouse | | | |

### Example 2

Influence of the prophylactic administration of the blood-lipid-lowering traditional Chinese medicine compositions on the rat model of lipid metabolism disorder.

Thirty SD rats were fed with general nutrition food for a week, then the blood collection was carried out via capillary eye canthus for separating serum. The levels of triglyceride (TG), total cholesterol (CHO), high density lipoprotein cholesterol (HDL-C) and low density lipoprotein cholesterol (LDL-C) were determined. According to the above respective item and the body weight index of rats, the animals were sampled and uniformly divided into five groups.

TABLE 4

| | | | | |
|---|---|---|---|---|
| 1 | Normal group: | n = 6 | General full | 0.5% CMC-Na solution |
| | | | nutrition food | 10 ml/kg |
| 2 | Model group: | n = 6 | High-fat food | 0.5% CMC-Na solution |
| | | | | 10 ml/kg |
| 3 | Positive group: | n = 6 | High-fat food | Atorvastatin 10 mg/ |
| | | | | 10 ml/kg |
| 4 | Low dose group: | n = 6 | High-fat food | Artesunate: ursolic acid |
| | | | | (25 + 25) mg/10 ml/kg |
| 5 | High dose | n = 6 | High-fat food | Artesunate: ursolic acid |
| | group: | | | (100 + 100) mg/10 ml/kg |

Two weeks after administration and modeling, the rats were anaesthetized with 12% chloral hydrate (0.3 ml/100 g). Then the abdominal aortic bloods were collected, the serum was centrifuging separated, and the levels of AST, ALT, ALP, T-Bil, TG, CHO, HDL-C and LDL-C were determined. Weighing the liver to calculate the liver coefficients. Fixing the main lobe of liver with 10% formalin, progressively dehydrating with ethanol, embedding in paraffin, slicing, HE conventional staining, making microscopy histological examination. Additionally, taking 400 mg of liver, homogenizing by adding 4 ml of lipid extract solution (heptane: isopropanol=2:3.5), oscillating for extracting the lipid, centrifugating, precipitating, and then taking the supernatant for determining the amounts of TG and CHO of liver with a kit.

Statistic analysis: All of the above indicators are expressed in mean±standard deviation, and compared using the t-test.

Experimental Results

1. The Influences of Compound Blood-Lipid-Lowering Drugs on the Serum CHO and TG of Rat Model

The results are shown in Table 4. The results illustrated that, after 2 weeks of high-fat food feeding, the serum CHO and TG of the model rat group were significantly higher than that before administration, which indicated the success of modeling high-lipid. Both the oral administrations of positive drug atorvastain of 10 mg/kg and the compound blood-lipid-lowering drugs of 50 and 200 mg/kg could significantly reduce the serum CHO level of hyperlipidemia rats, the difference between the positive drug and the model group had a significance (P<0.05), and the difference between the compound blood-lipid-lowering drugs of low or high dose group and the model group had an obvious significance (P<0.01).

2. The Influences of Prophylactic Administrations of Compound Blood-Lipid-Lowering Drugs on the Lipoprotein Cholesterol Content of the Lipid Metabolism Disorder Rat Model

The results were shown in Table 5. The results illustrated that, after 2 weeks of high-fat food feeding, there was no significant influence on HDL-C, but LDL-C was significantly higher than that before modeling, and the H/L ratios were significantly reduced. Both oral administrations of the positive drug atorvastain of 10 mg/kg and the compound blood-lipid-lowering drugs of 50 and 200 mg/kg could significantly increase the serum HDL-C levels of hyperlipidemia rats, wherein the difference between the positive drug and the model group had a significance (P<0.05), and the difference between the compound blood-lipid-lowering drugs of low or high dose group and the model group had a obvious significance (P<0.01). As compared with the model group, the positive drug atorvastain had a significant effect of reducing LDL-C (P<0.05), while the compound blood-lipid-lowering drugs of both low dose group and high dose group could significantly reduce the serum LDL-C levels (P<0.01). The H/L results showed that, as compared with model group, the H/L ratios of animals of atorvastain and the compound blood-lipid-lowering drugs of low and high dose group significantly increased (P<0.01).

3. The Influences of Prophylactic Administrations of Compound Blood-Lipid-Lowering Drugs on the Liver Weight, Liver Index and Liver Lipid Content of the Lipid Metabolism Disorder Rat Model

The results were shown in Table 6. The results illustrated that, after 2 weeks of high-fat food feeding, the liver index, and CHO content and TG content in the liver tissue were significantly increased as compared with the normal group. As compared with the model group, the compound high-dose group could significantly reduce the liver weight, liver index and liver tissue CHO and TG levels (P<0.01), while the liver weight, liver index and liver lipid content of the other animal groups after administration and modeling had no significant difference as compared with the model group (P>0.05).

4. The Influences of Prophylactic Administrations of Compound Blood-Lipid-Lowering Drugs on the General Condition and Body Weight of Rats

Before the modeling, rats were normal in the appearance sign, behavior, mental state, food, drinking water and gland secretion. After the modeling and administration, the animals of the compound high-dose blood-lipid-lowering drug group became the states of having pale dirty yellow hair, reduced food intake, and obvious body weight reduction as compared with the states of the model group before administration (P<0.01). While animals of other groups were normal in general conditions after modeling and administration, and their body weight had no obvious difference as compared with the model group (P>0.05). The results were shown in Table 7.

5. The Influences of Prophylactic Administrations of Compound Blood-Lipid-Lowering Drugs on the Blood Biochemical Indices of the Lipid Metabolism Disorder Rat Model

The results were shown in Table 8. As compared with the normal group, the ALT, AST and ALP of the model group were significantly increased. As compared with the model group, the serum ALP of the compound low-dose blood-lipid-lowering drug group was significantly lower than that of the model group, while the AST and ALP of the compound high-dose blood-lipid-lowering drug group were also significantly lower, even much lower than that of the model group.

TABLE 4
The influences of prophylactic administrations of compound blood-lipid-lowering drugs
on the blood lipid content of the lipid metabolism disorder rat model

| | | | | CHO after 2 | | TG after 2 |
|---|---|---|---|---|---|---|
| | | | | weeks of | | weeks of |
| | | | CHO before | modeling and | TG before | modeling and |
| | Dose | | administion | administration | administion | administration |
| Group | mg/kg | N | mmol/L | mmol/L | mmol/L | mmol/L |
| Normal | / | 6 | 1.56 ± 0.23 | 1.26 ± 0.08** | 1.50 ± 0.61 | 0.45 ± 0.24** |
| Model | / | 6 | 1.53 ± 0.26 | 4.46 ± 0.80 | 1.52 ± 0.32 | 2.22 ± 0.87 |
| Atorvastatin | 10 | 6 | 1.61 ± 0.35 | 3.35 ± 0.50* | 1.52 ± 0.33 | 2.14 ± 1.03 |
| Compound of | 50 | 6 | 1.48 ± 0.21 | 3.41 ± 0.39* | 1.46 ± 0.15 | 2.10 ± 1.04 |
| low dose | | | | | | |
| Compound of | 200 | 6 | 1.42 ± 0.29 | 3.01 ± 0.26** | 1.51 ± 0.41 | 1.35 ± 1.36 |
| high dose | | | | | | |

*P < 0.05,
**P < 0.01, as compared with the model group.

TABLE 5
The influences of prophylactic administrations of compound
blood-lipid-lowering drugs on the lipoprotein cholesterol content of the lipid metabolism
disorder rat model

| | | | HDL-C | HDL-C after 2 | LDL-C | LDL-C after 2 | | |
|---|---|---|---|---|---|---|---|---|
| | | | before | weeks of | before | weeks of | | H/L after 2 |
| | Dose | | administion | administration | administion | administration | H/L before | weeks of |
| Group | mg/kg | N | mmol/L | mmol/L | mmol/L | mmol/L | administion | administration |
| Normal | / | 6 | 0.69 ± 0.14 | 0.75 ± 0.11 | 0.49 ± 0.28 | 0.30 ± 0.11** | 2.77 ± 3.42 | 2.69 ± 0.83** |
| Model | / | 6 | 0.67 ± 0.09 | 0.72 ± 0.12 | 0.42 ± 0.22 | 2.40 ± 0.80 | 2.42 ± 2.13 | 0.33 ± 0.12 |
| Atorvastatin | 10 | 6 | 0.74 ± 0.06 | 1.04 ± 0.17** | 0.43 ± 0.24 | 1.27 ± 0.47* | 2.35 ± 1.44 | 0.90 ± 0.30** |
| Compound | 50 | 6 | 0.68 ± 0.05 | 1.05 ± 0.13** | 0.38 ± 0.15 | 1.04 ± 0.29** | 2.08 ± 1.01 | 1.09 ± 0.37** |
| of low dose | | | | | | | | |
| Compound | 200 | 6 | 0.68 ± 0.09 | 1.10 ± 0.10** | 0.31 ± 0.13 | 0.40 ± 0.24** | 2.45 ± 0.86 | 3.86 ± 2.30* |
| of high dose | | | | | | | | |

*P < 0.05,
**P < 0.01, as compared with the model group.

TABLE 6
The influences of prophylactic administrations of compound
blood-lipid-lowering drugs on the liver weight, liver index and liver lipid content of the lipid
metabolism disorder rat model

| | Dose | | | Liver index | CHO content of liver | TG content of liver |
|---|---|---|---|---|---|---|
| Group | mg/kg | N | Liver weight g | mg/g | mmol/L | mmol/L |
| Normal | / | 6 | 7.77 ± 0.42** | 28.92 ± 0.97** | 1.93 ± 0.52** | 4.95 ± 0.85** |
| Model | / | 6 | 11.19 ± 0.80 | 42.51 ± 1.80 | 9.00 ± 1.19 | 10.76 ± 1.09 |
| Atorvastatin | 10 | 6 | 11.06 ± 0.77 | 41.49 ± 1.97 | 9.66 ± 1.23 | 10.37 ± 1.72 |
| Compound of low dose | 50 | 6 | 11.36 ± 0.94 | 43.89 ± 1.71 | 8.24 ± 1.07 | 9.99 ± 0.81 |
| Compound of high dose | 200 | 6 | 7.74 ± 0.86** | 37.04 ± 2.34** | 5.67 ± 1.77** | 7.07 ± 1.69** |

*P < 0.05,
**P < 0.01, as compared with the model group.

TABLE 7
The influences of prophylactic administrations of compound blood-lipid-
lowering drugs on the body weight of the lipid metabolism disorder
rat model

| | | | After 1 | After 2 | After 3 |
|---|---|---|---|---|---|
| | Dose | | week of | weeks of | weeks of |
| | mg/ | | administr- | administration | administration |
| Group | kg | N | ation g | g | g |
| Normal | / | 6 | 227.5 ± 8.2 | 253.8 ± 11.1 | 268.7 ± 10.4 |
| Model | / | 6 | 229.7 ± 9.4 | 247.3 ± 13.5 | 263.0 ± 11.6 |
| Atorvastatin | 10 | 6 | 231.2 ± 7.6 | 255.0 ± 11.0 | 266.7 ± 13.7 |
| Compound | 50 | 6 | 230.3 ± 8.8 | 243.0 ± 12.2 | 258.7 ± 17.2 |
| of low dose | | | | | |
| Compound | 200 | 6 | 234.3 ± 13.6 | 207.5 ± 12.9** | 208.8 ± 17.6** |
| of high dose | | | | | |

*P < 0.05,
**P < 0.01, as compared with the model group.

TABLE 8
The influences of prophylactic administrations of compound blood-lipid-lowering
drugs on the blood biochemical indices of the lipid metabolism disorder rat model

| | Dose | | | | | |
|---|---|---|---|---|---|---|
| Group | mg/kg | N | ALT IU/L | AST IU/L | ALP IU/L | T-Bil μmol/L |
| Normal | / | 6 | 21.0 ± 5.9** | 70.2 ± 4.2** | 21.8 ± 3.7** | 6.84 ± 1.93 |
| Model | / | 6 | 49.2 ± 9.5 | 97.3 ± 17.5 | 48.5 ± 6.3 | 5.30 ± 0.71 |
| Atorvastatin | 10 | 6 | 52.7 ± 12.5 | 94.3 ± 30.1 | 43.2 ± 8.8 | 6.61 ± 2.57 |
| Compound of low dose | 50 | 6 | 58.3 ± 15.9 | 76.5 ± 42.4 | 34.7 ± 9.9* | 7.98 ± 2.88 |
| Compound of high dose | 200 | 6 | 66.0 ± 25.7 | 75.2 ± 16.8* | 16.5 ± 9.6** | 5.28 ± 1.72 |

*P < 0.05,
**P < 0.01, as compared with the model group.

### Example 3

### The Preparation Method of Tablets of a Blood-Lipid-Lowering Traditional Chinese Medicine Composition

Weighing 200 g of artesunate and 200 g of ursolic acid, comminuting them at a weight ratio of 1:1 to be a uniform powder mixture, then screening through a 120 mesh sieve. Adding 40 g of hydroxypropyl methyl cellulose, 100 g of microcrystalline cellulose and 15 g of cross-linked sodium carboxymethyl cellulose, mixing uniformly, then adding an appropriate amount of 60% (v/v) ethanol solution so as to form a soft material, wherein said appropriate amount refers to an ethanol amount ensuring to reach the standard of forming a soft material. Screening through a 24 mesh sieve, granulating, drying at 50 for 2 hours, screening the dried particles through a 30 mesh sieve for pelletizing. Adding 2.5 g of magnesium stearate, mixing, and then tabletting.

What is claimed:

1. A blood-lipid-lowering traditional Chinese medicine composition, characterized in comprising artemisinin derivatives API and ursolic acid API, wherein the weight ratio of artemisinin derivatives API to ursolic acid API is from 1:25 to 25:1.

2. The blood-lipid-lowering traditional Chinese medicine composition according to claim 1, characterized in that the artemisinin derivatives API is one selected from the group consisting of artemisinin, dihydroartemisinin, artesunate, artemether, and the combination of two or more thereof.

3. The blood-lipid-lowering traditional Chinese medicine composition according to claim 1, characterized that the ursolic acid API is ursolic acid.

4. The blood-lipid-lowering traditional Chinese medicine composition according to claim 1, 2 or 3, characterized that the weight ratio of artesunate API to ursolic acid API is 1:1.

5. A method for preparing the blood-lipid-lowering traditional Chinese medicine composition according to claim 1, characterized in comprising the following steps: mixing artemisinin derivative API and ursolic acid API at a weight percentage ratio; comminuting the mixture, screening through a 120 mesh sieve; then adding suitable accessories to get a clinically acceptable formulation; alternatively, the method comprises the following steps: weighing artemisinin derivative API and ursolic acid API at a weight percentage ratio; comminuting them respectively, screening through a 120 mesh sieve; then mixing and screening for three times to obtain the composition.
